(19) United States
(12) Patent Application Publication
(10) Pub. No.: US 2025/0020467 A1
(43) Pub. Date: Jan. 16, 2025

(54) MOTION CAPTURE SENSOR WITH COMPENSATION FOR DRIFT AND SATURATION

(71) Applicant: AIQ Synertial Ltd., Brighton (GB)

(72) Inventor: Edwin P. Berlin, Sebastopol, CA (US)

(21) Appl. No.: 18/762,310

(22) Filed: Jul. 2, 2024

Related U.S. Application Data
(60) Provisional application No. 63/513,068, filed on Jul. 11, 2023.

(51) Int. Cl. G01C 21/16

(52) U.S. Cl. CPC G01C 21/16

(57) ABSTRACT

An error-compensating inertial measurement unit includes a high dynamic range six degree of freedom inertial measurement unit and a nine degree of freedom inertial measurement unit, both strongly attached to a rigid substrate. A sensor processor determines a magnitude of mathematical difference between an orientation determined from the six degree of freedom inertial measurement unit and an orientation determined by a fusion processor in the nine degree of freedom measurement unit. When the magnitude of difference is greater than a sensor drift threshold, the absolute orientation of the six degree of freedom inertial measurement unit is modified to the orientation determined from the nine degree of freedom inertial measurement unit. When the nine degree of freedom inertial measurement unit estimates that its measurement accuracy is reduced by sensor saturation, modifying the absolute orientation of the six degree of freedom inertial measurement unit is delayed.

### FIELD OF THE INVENTION

Embodiments of the invention are related to motion capture sensors having an inertial measurement unit for measuring linear and angular acceleration.

### BACKGROUND

A mathematical model of the positions and movements of a motion capture subject may be formed from data collected from motion capture (mocap) sensors positioned on the motion capture subject. A mocap sensor may be described by a number of degrees of freedom measured by the sensor, wherein the number of degrees of freedom corresponds to the number of independent parameters measurable by the sensor. For example, a six degree of freedom (6-DOF) mocap sensor may include an inertial measurement unit (IMU) having accelerometers with three degrees of freedom configured to measure three linear acceleration components of motion relative to three orthogonal spatial axes and gyroscopes configured to measure three angular acceleration components of motion relative to the same three spatial axes. There may be, for example, one accelerometer and one gyroscope for each of the three spatial axes for a total of six degrees of freedom. A nine degree of freedom (9-DOF) mocap sensor may include an IMU having three-degree-of-freedom (3-DOF) accelerometers, 3-DOF gyroscopes, and 3-DOF magnetometers. There may be, for example, one accelerometer, one gyroscope, and one magnetometer for each of the three spatial axes for a total of nine degrees of freedom. The magnetometers may be configured to measure rotational orientation of the sensor relative to the earth's magnetic field.

Inertial data measured by an IMU may be mathematically integrated over selected time intervals to calculate corresponding velocity and position values. However, integration adds offset terms to calculated results, causing cumulative errors between calculated velocities, angles, and positions, and actual values. Noise and measurement errors present in the inertial data may further add to the cumulative errors. The cumulative errors may cause substantial, observable differences between the mathematical model of the positions and angles of the mocap subject and the actual positions and angles of the mocap subject. Cumulative sensor errors may be referred to as sensor drift. For 6-DOF IMUs lacking internal sensor fusion capability, the longer the integration time interval, the greater the magnitude of sensor drift in data output from the IMU.

The dynamic range of an IMU may be expressed in terms of the minimum and maximum values measurable by the IMU and may further include parameters related to a minimum time duration needed to form and output each measured value and/or a maximum data rate at which data values may be output. When a mocap subject moves with sufficient acceleration to approach or exceed the dynamic range of an IMU, an error can develop between acceleration values measured by the IMU and actual accelerations experienced by the motion capture subject. When a change in mocap subject acceleration close to or greater than the IMU's dynamic range fails to produce a corresponding proportional change in data measured by the IMU, the IMU may be referred to as being saturated. A saturated IMU may produce an output value corresponding to a maximum acceleration value measurable by the sensor. A mathematical model using data from a saturated IMU may incorrectly represent actual mocap subject accelerations, positions, and spatial orientations.

Sensor drift and sensor saturation contribute to measurement errors which can be limitations for motion capture studies. For example, a motion capture study may be conducted to measure and improve an athlete's performance. It may be desirable to accurately determine relative to an athlete's body positions the spatial locations, spatial orientation angles, and rates of movement of an article of equipment such as a golf club, tennis racket, or baseball bat. Mocap sensors may be placed on the athlete's hand, forearm, and upper arm, to model the athlete's movements and on the athletic equipment at locations chosen to represent the spatial location, velocity, angular orientation, and/or acceleration of the equipment at selected sample times. Angular orientation may also be referred to as attitude. Sensor drift leads to accumulating errors in the modelled positions and movements of the athlete's body and sports equipment. Furthermore, some locations on an article of athletic equipment may have substantially larger displacements, angular acceleration, and/or linear acceleration than the athlete's hands or other parts of an athlete's body, increasing the possibility of saturation for mocap sensors attached to the equipment.

Sensor saturation is not limited to measurements made by mocap sensors attached to sports equipment. For example, a 9-DOF IMU may saturate during measurements of the positions, velocities, accelerations, and spatial angles (orientation) of a baseball pitcher's arm, hand, and fingers while the pitcher throws a fastball. A 6-DOF IMU with a sufficiently large dynamic range may not saturate during the pitch but may accumulate sufficient measurement error over time to make it difficult to compare one pitch to another. Sensor saturation may also result in unwanted measurement errors in mocap data collected for motion capture activities related to medical procedures such as surgery or physical therapy and mocap models created for video games, motion pictures, musical performances, ergonomic studies, and other applications.

### SUMMARY

An example apparatus embodiment of an error-compensating inertial measurement unit (ECIMU) includes a nine degree of freedom inertial measurement unit (9-DOF IMU) connected for data communication with a sensor processor. The ECIMU further includes a high dynamic range six degree of freedom inertial measurement unit (6-DOF IMU) connected for data communication with the sensor processor. The 9-DOF IMU includes a three-degree of freedom (3-DOF) accelerometer, a 3-DOF gyroscope, a 3-DOF magnetometer, a fusion processor connected for signal communication with the 3-DOF accelerometer, the 3-DOF gyroscope, and the 3-DOF magnetometer, with the fusion processor configured in some embodiments to output inertial measurement data in quaternion form. The 9-DOF IMU further includes an accuracy estimator operated by the fusion processor. The accuracy estimator is configured to form an accuracy estimate for the 9-DOF IMU. The six degree of freedom inertial measurement unit (6-DOF IMU) preferably consists of a high dynamic range 3-DOF accelerometer and a high dynamic range 3-DOF gyroscope.

The example sensor processor is preferably configured to determine a first orientation by integration of data from the 6-DOF IMU; determine a second orientation from the inertial measurement data in quaternion form from the 9-DOF IMU; and estimate a magnitude of sensor drift by forming a magnitude of mathematical difference between the first orientation and the second orientation. The sensor processor is further configured to compare the estimate of sensor drift to a sensor drift threshold value stored in a nonvolatile memory included in the ECIMU. When the estimate of sensor drift is less than the sensor drift threshold value, the sensor processor is configured to output data in quaternion form from the 6-DOF IMU on a serial data output terminal. When the estimate of sensor drift is greater than the sensor drift threshold value and the accuracy estimate for the 9-DOF IMU is less than a value corresponding to sensor saturation, the sensor processor is configured to set an absolute orientation of the 6-DOF IMU to the second orientation.

The example apparatus embodiment of an ECIMU further preferably includes a rigid substrate, with the 9-DOF IMU strongly attached to the rigid substrate and the 6-DOF IMU strongly attached to the rigid substrate.

The example accuracy estimator is configured to form the accuracy estimate from a value selected from a first accuracy estimate corresponding to sensor saturation, a second accuracy estimate corresponding to a value of measurement error less than a value of sensor saturation, and a third accuracy estimate corresponding to the value of measurement error less than an error threshold value. In an example embodiment, the first accuracy estimate is preferably larger in magnitude than the second accuracy estimate and the second accuracy estimate is preferably larger in magnitude than the third accuracy estimate.

The example ECIMU further includes a serial data input terminal electrically connected to the sensor processor and a serial data output terminal electrically connected to the sensor processor, with the sensor processor configured to output on the serial data output terminal the inertial measurement data from the 6-DOF IMU having a magnitude of sensor drift less than the sensor drift threshold.

An example apparatus embodiment of an error-compensating motion capture system includes a first ECIMU and a second ECIMU connected for data communication with the first ECIMU with the serial data input terminal of the first ECIMU connected to the serial data output terminal of the second ECIMU, thereby forming a series-connected set of ECIMU devices. The series-connected set optionally includes an additional plurality of ECIMU devices electrically connected in series to one another and to the second ECIMU.

The example error-compensating motion capture system optionally further includes a hub having a hub processor and a hub nonvolatile memory connected for data communication with the hub processor. The serial data output terminal of the first ECIMU in the series-connected set is connected for data communication with an input of the hub processor. A sensor command line connected to the hub processor is further connected to a command input terminal of each ECIMU in the error-compensating motion capture system.

### BRIEF DESCRIPTION OF THE DRAWINGS

FIG. 1 is a schematic diagram of an example apparatus embodiment of an error-compensating inertial measurement unit (ECIMU).

FIG. 2 is a schematic diagram representing examples of an IMU first orientation determined from the HDR 6-DOF IMU in the ECIMU, a IMU second orientation determined from the 9-DOF IMU in the ECIMU, and an angular orientation difference, representing an example of a magnitude of sensor drift.

FIG. 3 is a schematic diagram of some parts of an example error-compensating motion capture apparatus for measuring and recording movements of a motion capture subject with ECIMUs in accord with the example of FIG. 1.

FIG. 4 is a block diagram representing an example of a series-sequential set of data packets formed by the example error-compensating motion capture system of FIG. 3.

### DESCRIPTION

Embodiments of a motion capture (mocap) system include an error-compensating inertial measurement unit (ECIMU) with a nine degree of freedom (9-DOF) inertial measurement unit (IMU) and a high dynamic range (HDR) six degree of freedom (6-DOF) IMU connected for data communication with a sensor processor in the ECIMU. The sensor processor uses the six degree of freedom inertial measurements from the HDR 6-DOF IMU to continuously update a quaternion representation of the orientation of the sensor. When the sensor processor receives accurate orientation measurement data in quaternion form from the 9-DOF IMU, the sensor processor applies that orientation as the absolute orientation of the 6-DOF quaternion, thereby nulling sensor drift error accumulated in the 6-DOF IMU data since the previous compensation from 9-DOF data. After being adjusted for accumulated sensor drift, the 6-DOF IMU quaternion output from the 6-DOF IMU may be referred to as an adjusted quaternion. The absolute orientation of the 6-DOF IMU is the reference orientation from which subsequent orientation changes are measured until the next compensation is performed. The adjusted 6-DOF IMU quaternion forms the output data from the ECIMU.

The sensor processor performs mathematical integration of quaternions from the HDR 6-DOF IMU and monitors the orientation calculated from integration of the HDR 6-DOF IMU quaternions. Orientation corresponds to angles of rotation in three spatial dimensions with respect to three mutually orthogonal spatial axes. Sensor drift of the HDR 6-DOF IMU leads to a cumulative difference between the orientation calculated from integration of the HDR 6-DOF IMU data and the orientation determined from the 9-DOF IMU data. The sensor processor optionally interrogates the 9-DOF IMU at regular time intervals to refresh orientation information, compares the orientation from the 9-DOF IMU to the orientation computed by integration of the HDR 6-DOF data, and computes an orientation error magnitude from the mathematical differences between the orientation from the 9-DOF IMU and the orientation computed by integration of data from the HDR 6-DOF IMU.

At each reading of quaternions from the 9-DOF IMU, the sensor processor also receives a value representing an estimate of the accuracy of the estimated orientation quaternion of the 9-DOF IMU. Saturation is one of several possible sources of error in the estimated orientation quaternion. When the estimate of accuracy for the 9-DOF IMU indicates saturation and other errors are acceptably small in measured 9-DOF data, and when the computed orientation difference between 9-DOF data and 6-DOF data is greater than or equal to a specified sensor drift threshold value for the HDR 6-DOF IMU, the sensor processor recalibrates the HDR 6-DOF IMU with the latest orientation angles from the 9-DOF IMU to reduce sensor drift errors. However, when the estimate of accuracy indicates sensor saturation and/or other error effects may be present in data from the 9-DOF IMU, the sensor processor delays recalibration of the HDR 6-DOF IMU until the next time interval when the estimate of accuracy for the 9-DOF IMU indicates saturation and/or other errors are unlikely to be significant in acceleration measurements by the 9-DOF IMU.

The disclosed example ECIMU embodiments are effective for accurately removing sensor drift contributions to acceleration data output from the HDR 6-DOF IMU at each time interval for which data from the 9-DOF IMU is not affected by measurements errors, and for providing potentially acceptable data from the HDR 6-DOF IMU when measurement errors of the 9-DOF IMU have been estimated or detected to be present. The combination of the 9-DOF IMU, HDR 6-DOF IMU, and sensor processor minimizes sensor drift in quaternions output from the ECIMU even when movements by a mocap subject exceed the maximum acceleration measurable by the 9-DOF IMU, with less sensor drift over the full duration of a movement by a mocap subject than would occur with an uncompensated HDR 6-DOF IMU.

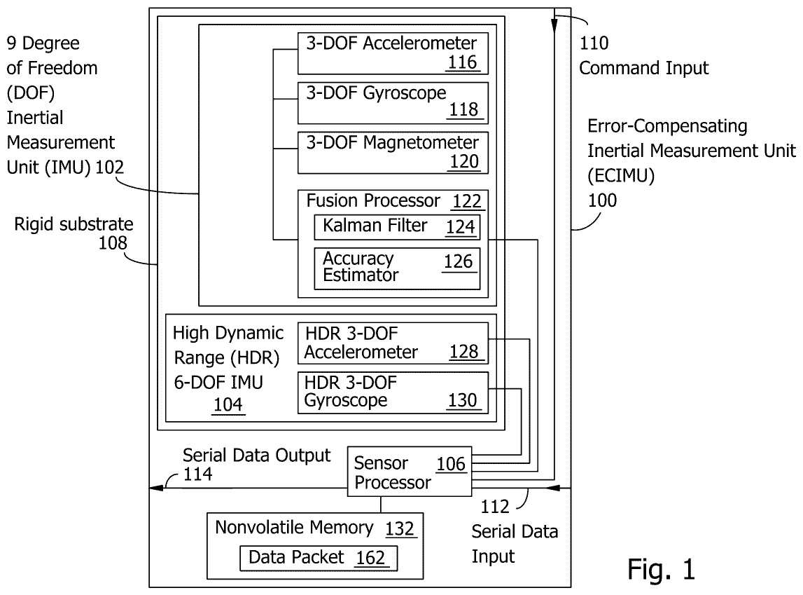

An example ECIMU 100 device embodiment is shown as a schematic diagram in FIG. 1. The example ECIMU 100 includes a 9-DOF IMU 102 and an HDR 6-DOF IMU 104 electrically connected for data communication with a sensor processor 106. The 9-DOF IMU 102 and the HDR 6-DOF IMU 104 are both strongly attached to a rigid substrate 108. The substrate is preferably sufficiently rigid to prevent a magnitude of relative motion between the 9-DOF IMU and 6-DOF IMU that is greater than the minimum detection thresholds for the IMUs. Internal sensors in the HDR 6-DOF IMU 104 include an HDR 3-DOF accelerometer 128 and an HDR 3-DOF gyroscope 130, with each internal sensor electrically connected for data communication with the sensor processor 106. Internal sensors in the 9-DOF IMU 102 includes a 3-DOF accelerometer 116, a 3-DOF gyroscope 118, and a 3-DOF magnetometer 120 each electrically connected for data communication with a fusion processor 122.

The fusion processor 122 in the 9-DOF IMU 102 combines data from the accelerometers 116, gyroscopes 118, and magnetometers 120 to form 9-DOF IMU output inertial measurement data having greater accuracy than may be obtained from any one of the individual internal sensors. Combining data from the internal sensors to form IMU output quaternions may be referred to as sensor fusion. Some embodiments of the example fusion processor 122 in the 9-DOF IMU 102 optionally further include implementations of a Kalman filter 124 and an accuracy estimator 126. The Kalman filter 124 may be used, for example, to estimate parameter values and to reduce the effects of noise and measurement errors during sensor fusion. The accuracy estimator 126 produces an estimate of measurement accuracy from saturation and other error sources. In some embodiments the estimate of sensor accuracy is represented by a first accuracy estimate, a second accuracy estimate, and an optional third accuracy estimate for inertial measurement data output from the 9-DOF IMU. For example, a first accuracy estimate with numerical value 0 may represent saturated sensor data that is not changing in response to acceleration changes imposed on the sensor. A second accuracy estimate with a numerical value of 1 may represent that saturation or other substantial measurement errors may be contributing to inertial data measurements, for example lower-order bits in measured inertial data values not changing from one sample to the next. A third accuracy estimate with a numerical value of 2 may represent measurement error less than a specified error threshold. Accuracy estimates may have more than three discrete numerical values, each discrete value possibly corresponding to different specified thresholds of sensor measurement errors.

The high dynamic range 6-DOF IMU 104 preferably has a higher dynamic range than the 9-DOF IMU 102 and can preferably detect substantially faster rates of change in inertial measurements than the 9-DOF IMU. For example, an HDR 6-DOF IMU used for mocap studies of athletic performance may be able to measure changes in orientation at about 4000 degrees per second. The 9-DOF IMU in the same ECIMU 100 may have a substantially smaller dynamic range, for example half the dynamic range of the HDR 6-DOF IMU. The 9-DOF IMU may operate with substantially longer time intervals between inertial measurements than the HDR 6-DOF IMU.

Calibration information for the HDR 6-DOF IMU 104 determined by the sensor processor 106 from data obtained from quaternions output by the 9-DOF IMU 102 may be stored in the ECIMU 100 in a nonvolatile memory 132 electrically connected for data communication with the sensor processor. Quaternions from the HDR 6-DOF IMU may also be stored in the nonvolatile memory 132 in the ECIMU 100 until the sensor processor 106 is directed to transmit a data packet 162 including the inertial measurements on the serial data output terminal to another device. The data packet 162 from the HDR 6-DOF IMU may optionally be combined by the sensor processor 106 with data received on the serial data input terminal 112 from another ECIMU 100 when such input data is available. Examples of other parameters optionally stored in the nonvolatile memory 132 include, but are not limited to, a data accuracy estimate from the accuracy estimator 126, a first accuracy estimate value corresponding to sensor saturation, a second accuracy estimate value corresponding to a measurement error less than sensor saturation, a third accuracy estimate value less than an error threshold, the error threshold, a first orientation determined from the HDR 6-DOF IMU, a second orientation determined from the 9-DOF IMU, an orientation difference between the first orientation and second orientation, a sensor drift threshold, data in quaternion form from the 9-DOF IMU, adjusted quaternion data for the 6-DOF IMU, and a sequential set of data packets 164 received from another ECIMU 100.

Data interpolation may be used by the sensor processor 106 in the ECIMU 100 to compare data from the 9-DOF IMU 102 to data from the HDR 6-DOF IMU 104 and to form correction values for the HDR 6-DOF IMU from values obtained from the 9-DOF IMU. Data interpolation may be implemented as linear interpolation, spherical linear interpolation in which motion is assumed constant for a selected time interval along a great-circle path, or other interpolation techniques. A Kalman filter may be employed to produce estimates of sensor values and/or sensor errors.

The example ECIMU 100 of FIG. 1 includes at least two input terminals configured to receive information in serial form, including a command input terminal 110 electrically connected to the sensor processor 106 and a serial data input terminal 112 electrically connected to the sensor processor. The example ECIMU 100 further includes a serial data output terminal 114 connected to the sensor processor. Examples of commands which may be received by the sensor processor on the command input terminal 110 include, but are not limited to, an acquisition trigger command to start data recording, extraction of orientation angles from 9-DOF quaternions, transfer of orientation angles from the 9-DOF IMU to the HDR 6-DOF IMU to initiate a recalibration and/or compensation for sensor drift, and initiate storage of quaternions from the HDR 6-DOF IMU in the nonvolatile memory 132.

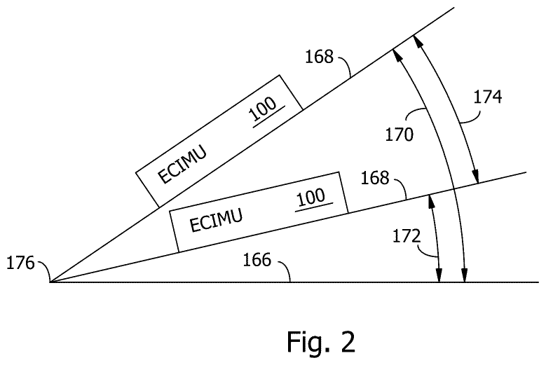

A schematic representation of examples of angular orientations determined by the 9-DOF IMU 102 and the HDR 6-DOF IMU 104 in an ECIMU 100 are shown in FIG. 2. The ECIMU 100 in the illustrated example has measured with its internal 6-DOF IMU 104 a first orientation angle 170 between a reference axis 168 of the ECIMU 100 and an external spatial reference axis 166. Concurrently with the 6-DOF IMU measurement, the same ECIMU 100 has measured with its internal 9-DOF IMU 102 a second orientation angle 172 between the reference axis of the ECIMU 100 and the external spatial reference axis 166. The first orientation angle 170 and second orientation angle 172 may be measured with respect to a center of rotation 176 for the actual angular displacement of the ECIMU 100, for example an angular displacement caused by movement of a mocap subject. The sensor processor 106 is configured to determine an angular orientation difference 174 between the first orientation angle 170 and the second orientation angle 172 and apply the angular orientation difference 174 to adjustment of HDR 6-DOF quaternions as previously described. An angular orientation 174 may be determined for any selected reference axis 168 of an ECIMU 100 and any external spatial reference axis 166, and adjusted quaternions determined for the HDR 6-DOF IMU 104 for all such axes.

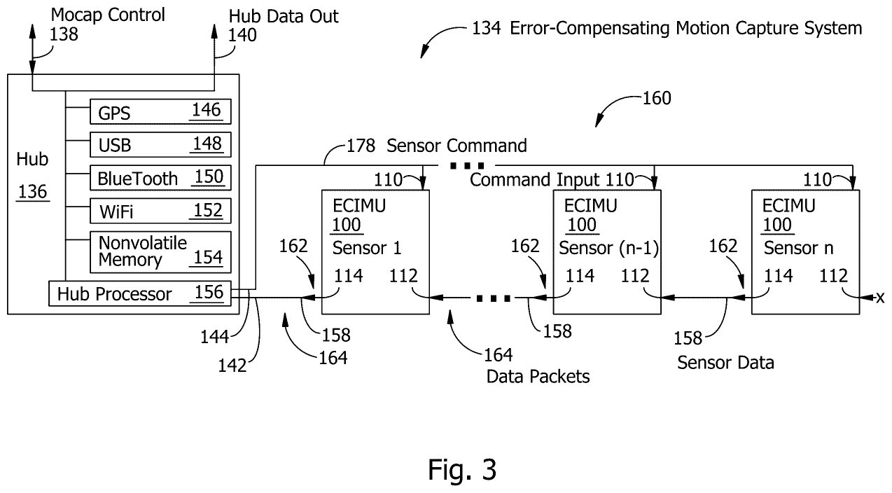

As suggested in the example of FIG. 3, some apparatus embodiments include more than one ECIMU 100 electrically connected in series for data communication with one another and with a hub 136. The example error-compensating motion capture system 134 of FIG. 3 includes a number “n” of ECIMU 100 devices electrically connected in series to one another and to a hub 136. Each of the n ECIMU devices in the series-connected set 160 may be placed on a motion capture subject at locations that are spatially separated from one another. A sensor command terminal 144 connected from a hub processor 156 in the hub 136 to the command input terminal 110 on each ECIMU 100 over an intervening sensor command line 178 provides for simultaneous triggering of all ECIMU devices to establish time correlation of data points from each ECIMU 100 with data points from other ECIMU devices in the error-compensating motion capture system 134. The nth ECIMU device (labelled Sensor n in FIG. 3) has its serial data output terminal 114 electrically connected by a sensor data line 158 to the serial data input terminal 112 of the next ECIMU (labelled sensor (n−1) in FIG. 3) in the series-connected set 160 of ECIMU 100 devices. Sensor (n−1) has its serial data output terminal 114 connected to the serial data input terminal 112 of the next upstream ECIMU by another sensor data line 158, with serial connections established output-to-input up to the first ECIMU device labelled Sensor 1 in FIG. 3. Sensor 1 has its serial data output terminal 114 connected for signal communication over a sensor data line 158 with the sensor data input terminal 142 of the hub processor 156. No other ECIMU 100 devices are electrically connected in series between Sensor 1 and the hub 136.

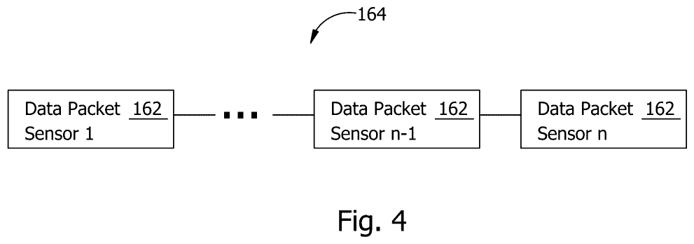

When the error-compensating motion capture system 134 is configured as shown in the example of FIG. 3 and is operating correctly, sensor n is the only ECIMU 100 with no other ECIMU 100 connected to its serial data input terminal 112. The ECIMU 100 is preferably configured to detect when no other ECIMU is connected to its serial data input. When the hub processor 156 sends a command to all ECIMU devices to initiate data acquisition and storage, sensor n initiates the formation of a sequential set of data packets 164, each ECIMU 100 forming its data packet 162 comprising measured quaternion data compensated for 6-DOF sensor drift and possibly for 9-DOF sensor saturation and appending to its data packet the sequence of data packets received from ECIMU units closer in the serial connection sequence 160 to sensor n, then transmitting the new sequential set 164 to the next sensor. As suggested in the example of FIG. 4, the sequential set of data packets 164 is complete after sensor 1 includes its data packet 162 with the set of n−1 preceding data packets originating at sensor n, after which sensor 1 forwards the complete set 164 of n data packets 162 to the hub processor 156. The sequential set of data packets 164 may alternatively be formed by each ECIMU 100 appending its data packet 162 to the end of the set of data packets 164 received from the preceding ECIMU in the series-connected set 160.

Each data packet added by an ECIMU 100 to the sequential set of data packets 164 includes inertial measurement data from the HDR 6-DOF IMU with sensor drift and saturation errors below the specified threshold values and/or inertial measurement data whose sensor drift errors have been adjusted as previously described by data from the 9-DOF IMU. The sequential set of data packets 164 including adjusted 6-DOF quaternions may be referred to as an error-compensated sequential set of data packets 164.

The hub 136 optionally includes a hub nonvolatile data memory 154 connected for data communication with the hub processor 156. The hub processor stores in the hub nonvolatile memory 154 the sequential set of error-compensated data packets 164 received on the sensor data input terminal 142 from sensor 1. The hub nonvolatile memory 154, hub processor 156, and hub communications capabilities enable the hub and the series-connected set 160 of ECIMU devices to optionally function as a stand-alone error-compensating motion capture system 134 operable without wired or wireless connection to another storage device. The hub processor 156 may receive instructions and/or requests from an external device on a mocap control terminal 138 to initiate mocap data acquisition, report ECIMU status, store ECIMU data packets in the hub nonvolatile memory 154, output data packets to another device, or perform other functions. Error-compensated mocap data packets in series-sequential order from sensor n to sensor 1 and status information about the ECIMU devices and hub may be output to another device on a hub data output terminal 140 electrically connected for data communication with the hub processor 156, the hub nonvolatile memory 154, and one or more of a universal serial bus (USB) controller 148, a Bluetooth™ radio-frequency transceiver 150, and/or a radio-frequency WiFi transceiver 152. The WiFi transceiver 152 may be configured to perform bidirectional wireless communications with a local area network. Quaternions and other data from the ECIMU devices may be modified by the hub processor 156 to include global positioning system (GPS) location data and/or timing data from an optional GPS receiver 146 in the hub.

The series-connected set 160 of ECIMU devices 100 in the example error-compensating motion capture system 134 of FIG. 3 are advantageous for organizing captured mocap data into data for child segments and data for parent segments in a mathematical model of a mocap subject. Sensor 1 may, for example, be placed on a proximal limb segment of a person to represent the parent segment of a mocap model. Sensor 2 may be placed more distally, for example on the subject's forearm to represent a child segment connected to the parent segment (upper arm). Sensor 3 may be placed on the back of the hand, sensor 4 on a finger, and so on, with sensor n possibly located at the most-distal location to be represented in the mocap model. Other arrangements of ECIMUs on a mocap subject may alternatively be used.

When the error-compensating motion capture system 134 is operating as described, the hub processor 156 will receive a set of n packets in serial data from sensor 1, with the set including one packet from each ECIMU 100 in the error-compensating motion capture system 134. However, a failure in an ECIMU 100 or a sensor data line 158 may prevent data packets from the point of failure out to the nth ECIMU from reaching the hub processor 156. The hub processor may count the number of received data packets and compare the count to a number of expected data packets to estimate where such an interruption has occurred. For example, in an error-compensating motion capture system 134 with five series-connected ECIMU 100 devices sending data packets to a hub 136, a failure in the fourth ECIMU or a failure in the sensor data line 158 between the third and fourth ECIMUs may cause the hub to receive data packets from ECIMUs 1, 2, and 3, but not from ECIMUs 4 and 5. For this example, the number of received packets is 3 and the number of expected packets is 5, and the hub processor can send an alert message informing a system operator to check for a failed ECIMU or interrupted electrical connections at ECIMU 4.

What is claimed is:

1. An error-compensating inertial measurement unit, comprising:
a nine degree of freedom inertial measurement unit (9-DOF IMU), comprising a three-degree of freedom (3-DOF) accelerometer, a 3-DOF gyroscope, a 3-DOF magnetometer, and a fusion processor connected for signal communication with said 3-DOF accelerometer, said 3-DOF gyroscope, and said 3-DOF magnetometer, with said fusion processor configured to output inertial measurement data in quaternion form;
said fusion processor including an accuracy estimator configured to form an accuracy estimate for said 9-DOF IMU;
a six degree of freedom inertial measurement unit (6-DOF IMU) consisting of a high dynamic range 3-DOF accelerometer and a high dynamic range 3-DOF gyroscope; and
a sensor processor connected for data communication with said 9-DOF IMU and said 6-DOF IMU, with said sensor processor configured to:
determine a first ECIMU orientation by integration of data from said 6-DOF IMU;
determine a second ECIMU orientation from said inertial measurement data in quaternion form from said 9-DOF IMU;
estimate a magnitude of sensor drift by forming a magnitude of mathematical difference between said first ECIMU orientation and said second ECIMU orientation;
when said estimate of sensor drift is less than a sensor drift threshold, output on a serial data output terminal data in quaternion form from said 6-DOF IMU; and
when said estimate of sensor drift is greater than said sensor drift threshold and said accuracy estimate for said 9-DOF IMU is less than a value corresponding to sensor saturation, set an absolute orientation of said 6-DOF IMU to said second orientation.

2. The apparatus of claim 1, further comprising:
a rigid substrate;
said 9-DOF IMU strongly attached to said rigid substrate; and
said 6-DOF IMU strongly attached to said rigid substrate.

3. The apparatus of claim 1, further comprising said accuracy estimator configured to form said accuracy estimate from a value selected from a first accuracy estimate corresponding to sensor saturation, a second accuracy estimate corresponding to a measurement error less than sensor saturation, and a third accuracy estimate corresponding to said measurement error less than an error threshold.

4. The apparatus of claim 3, wherein said first accuracy estimate is larger in magnitude than said second accuracy estimate and said second accuracy estimate is larger in magnitude than said third accuracy estimate.

5. The apparatus of claim 1, further comprising said 6-DOF IMU having a dynamic range greater than 4000 degrees per second for inertial measurements.

6. The apparatus of claim 5, further comprising a dynamic range of said 9-DOF IMU less than half of said dynamic range of said 6-DOF IMU.

7. The apparatus of claim 1, further comprising a nonvolatile memory connected for data communication with said sensor processor, said processor configured to store in said nonvolatile memory:
said inertial measurement data in quaternion form from said fusion processor; and
said measurement data from said 6-DOF IMU having a magnitude of sensor drift less than said sensor drift threshold.

8. The apparatus of claim 1, further comprising:
a serial data output terminal electrically connected to said sensor processor;
a serial data input terminal electrically connected to said sensor processor; and
said sensor processor configured to output on said serial data output terminal said inertial measurement data from said 6-DOF IMU having a magnitude of sensor drift less than said sensor drift threshold.

9. An error-compensating motion capture apparatus, comprising:
an error-compensating inertial measurement unit (ECIMU), comprising:
a nine degree of freedom inertial measurement unit (9-DOF IMU), comprising:
a three-degree of freedom (3-DOF) accelerometer; a 3-DOF gyroscope, and a 3-DOF magnetometer;
a fusion processor connected for signal communication with said 3-DOF accelerometer, said 3-DOF gyroscope, and said 3-DOF magnetometer, said fusion processor configured to output inertial measurement data in quaternion form, and said fusion processor including an accuracy estimator configured to form an accuracy estimate for said 9-DOF IMU;
a six degree of freedom inertial measurement unit (6-DOF IMU) consisting of a high dynamic range 3-DOF accelerometer and a high dynamic range 3-DOF gyroscope; and
a sensor processor connected for data communication with said 9-DOF IMU and said 6-DOF IMU, said sensor processor configured to:
determine a first orientation by integration of data from said 6-DOF IMU;
determine a second orientation from said inertial measurement data in quaternion form from said 9-DOF IMU;
estimate a magnitude of sensor drift by forming a magnitude of mathematical difference between said first orientation and said second orientation;
when said estimate of sensor drift is less than a sensor drift threshold, output on a serial data output terminal data in quaternion form from said 6-DOF IMU; and
when said estimate of sensor drift is greater than said sensor drift threshold and said accuracy estimate for said 9-DOF IMU is less than a value corresponding to sensor saturation, set an absolute orientation of said 6-DOF IMU to said second orientation;
said ECIMU is a first ECIMU; and
a second of said ECIMU connected to said first ECIMU with said serial data output terminal of said first ECIMU connected to said serial data input terminal of said second ECIMU.

10. The error-compensating motion capture apparatus of claim 9, further comprising an additional plurality of said ECIMU in series electrical connection with one another, said first ECIMU, and said second ECIMU.

11. The error-compensating motion capture apparatus of claim 9, further comprising:
each of said ECIMU further including a command input terminal electrically connected to said sensor processor; and
a hub having a hub processor connected for data communication with said serial data output terminal of said first ECIMU, and said hub processor further connected to a sensor command line electrically connected to said command input terminal of each ECIMU.

12. The error-compensating motion capture apparatus of claim 11, said hub further comprising a hub nonvolatile memory connected for data communication with said hub processor, said hub processor configured to store in said hub nonvolatile memory a sequential set of error-compensated data packets received from said first ECIMU.

13. The error-compensating motion capture apparatus of claim 12, said sequential set of error-compensated data packets including inertial measurement data from each of said first ECIMU and said second ECIMU.

14. The error-compensating motion capture apparatus of claim 11, said hub further comprising a global positioning system receiver connected for data communication with said hub processor.

15. The error-compensating motion capture apparatus of claim 11, said hub further comprising a universal serial bus controller connected for data communication with said hub processor.

16. The error-compensating motion capture apparatus of claim 11, said hub further comprising a Bluetooth transceiver connected for data communication with said hub processor.

17. The error-compensating motion capture apparatus of claim 11, said hub further comprising a WiFi transceiver connected for data communication with said hub processor.
